(19) United States
(12) Patent Application Publication
(10) Pub. No.: US 2025/0349423 A1
(43) Pub. Date: Nov. 13, 2025

(54) SYSTEMS AND METHODS FOR THE DETECTION OF AUTISM BASED ON SPEECH-BASED BIOMARKERS

(71) Applicant: Modality.AI, Inc., San Francisco, CA (US)

(72) Inventors: Hardik Kothare, Burlingame, CA (US); Michael Neumann, Waiblingen (DE); William Burke, Portland, OR (US); Doug Habberstad, Savannah, GA (US); Jackson Liscombe, Mill River, ME (US); Oliver Roesler, Syke (DE); David Suendermann-Oeft, San Francisco, CA (US); David Paulter, San Francisco, CA (US); Andrew Cornish, Soutland (NZ); Vikram Ramanarayanan, San Francisco, CA (US)

(21) Appl. No.: 18/660,816

(22) Filed: May 10, 2024

(51) Int. Cl. G16H 50/20; G16H 40/67

(52) U.S. Cl. CPC G16H 50/20; G16H 40/67

(57) ABSTRACT

A system for remotely determining the potential presence of autism spectrum disorder in a user. The system includes a virtual agent that administers one or more tasks to the user. The user performs the tasks and the performance is captured by a camera. The captured audiovisual data is sent to a server that derives objective metrics which are then applied to a classifying algorithm. If certain metrics meet certain thresholds, the system determines that the user is exhibiting autism symptoms.

### FIELD OF THE INVENTION

The field of the invention is autism detection technologies.

### BACKGROUND

The background description includes information that may be useful in understanding the present invention. It is not an admission that any of the information provided herein is prior art or relevant to the presently claimed invention, or that any publication specifically or implicitly referenced is prior art.

Autism Spectrum Disorder (ASD) is said to be the fastest growing neurodevelopmental disorder globally. ASD is a neurodevelopmental disorder that has a severe impact on the quality of life of individuals through deficits in communicative abilities. A multi-site epidemiological survey estimates that one in 44 children (2.3%) aged 8 years has ASD in the United States. Prevalence estimates for adults with ASD in the US are currently unknown but a recent simulation study suggests that prevalence stands at 2.21% in US adults aged 18 and older. In England, this figure is estimated to be around 9.8 to 11 per 1,000 individuals. However, this estimate varies across world regions with the median prevalence at 100 per 10,000 people.

Thus, it is extremely important to develop and validate technological tools that can be used to monitor biomarkers related to ASD and the effects of potential therapeutic interventions in the future. A pervasive developmental deficit observed in individuals with ASD is general motor abnormalities with up to 79% of the ASD population thought to be affected. Particularly, previous studies have shown that children with autism exhibit difficulties in articulatory control and motor speech abilities.

Even with the current work in the field, there is a greater need for understanding this disorder and how to accurately determine the possibility that a patient may be showing signs of ASD.

Thus, there is still a need for a system that facilitates a detection of possible presence of autism that can be remotely administered.

### SUMMARY OF THE INVENTION

The inventive subject matter provides apparatus, systems and methods in which one or more tasks can be administered remotely via a virtual agent presented and executed on a user's computing device, such that a possible presence of autism can be detected.

In embodiments of the inventive subject matter, the virtual agent presents at least one task for the user to perform. The computing device captures audiovisual data of the performance of the task by the user and streams the audiovisual data to one or more remote computer devices.

The inventive subject matter includes a user's computing device through which a virtual agent presents one or more tasks for the user to perform. Using a camera and a microphone integral to or connected with the user's computing device, the user's performance is captured and audiovisual data of the performance is produced. The audiovisual data is then provided to at least one remote computing device. The audiovisual data can be streamed or otherwise transmitted to the remote computing device over a network such as the internet.

Upon receiving the audiovisual data, the remote computing device segments the data and calculates objective metrics for the use based on the segmented audiovisual data.

The remote computing device then applies a classifying algorithm to the objective metrics, through which the remote computing device can determine whether the user may have autism spectrum disorder based on the output of the classifying algorithm.

In embodiments of the inventive subject matter, the objective metrics are derived according to one or more of a speech acoustic domain, a facial domain, a linguistic domain, a cognitive domain a motor domain, and an emotional domain.

In embodiments of the inventive subject matter, the tasks presented via the virtual agent for the user to perform can include one or more of a counting task, a reading task (e.g., reading sentences, reading consonant-vowel-consonant words, etc.), an oral diadochokinesis task, a picture description task, a spontaneous speech task, a forward-and-backward digit span task, a word recall task, a semantic fluency task or a sequential command task.

In embodiments of the inventive subject matter, the remote computing device refines the objective metrics by first removing any objective metric values beyond a predefined number of standard deviations (e.g., five standard deviations) and then, for the remaining objective metrics, recalculating a mean and a standard deviation and removing any remaining objective metric values beyond a second number of standard deviations (e.g., three standard deviations).

The remote computing device then applies a classifying algorithm to the objective metrics to determine whether a user is exhibiting symptoms of autism spectrum disorder. These results can be returned to the user's computing device and/or sent to a healthcare provider, sponsor, clinician, or other caregiver or involved party.

Various objects, features, aspects and advantages of the inventive subject matter will become more apparent from the following detailed description of preferred embodiments, along with the accompanying drawing figures in which like numerals represent like components.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### BRIEF DESCRIPTION OF THE DRAWING

FIG. 1 is a diagrammatic overview of a system according embodiments of the inventive subject matter.

FIG. 2 is a flowchart illustrating the overall process executed by the system, according to embodiments of the inventive subject matter.

FIG. 3 provides a flowchart of the administration of the task.

FIG. 4 is a flowchart showing step 240 in detail.

FIG. 5 illustrates the process of refining the objective metrics.

FIG. 6 shows a table with some example metrics that can be derived by the server.

FIG. 7 illustrates the execution of the classifying algorithm.

### DETAILED DESCRIPTION

Throughout the following discussion, numerous references will be made regarding servers, services, interfaces, engines, modules, clients, peers, portals, platforms, or other systems formed from computing devices. It should be appreciated that the use of such terms, is deemed to represent one or more computing devices having at least one processor (e.g., ASIC, FPGA, DSP, x86, ARM, ColdFire, GPU, multi-core processors, etc.) programmed to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable media storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, the Internet, LAN, WAN, VPN, or other type of packet switched network.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

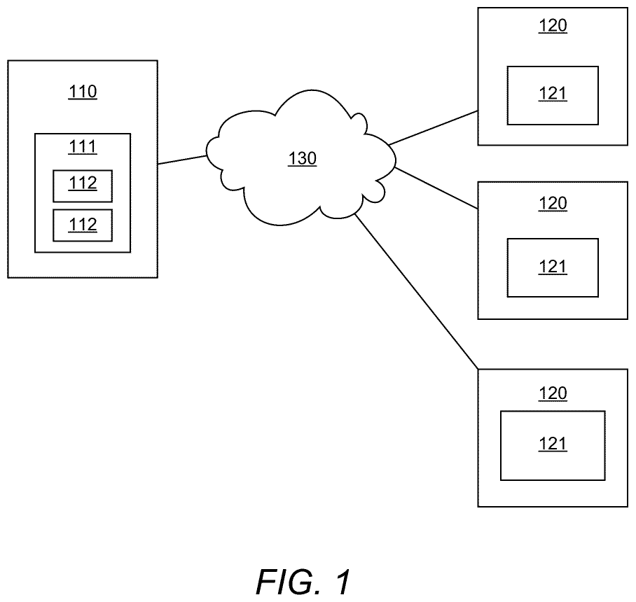

FIG. 1 is a diagrammatic overview of the system 100, according to embodiments of the inventive subject matter.

The system 100 includes a remote computing device 110 (otherwise referred to server 110) that can communicate to one or more client devices 120 over a network 130 (e.g., the internet). The server 110 can be one or more computing devices that include at least one processor, storage, and communication interface(s), located in one or more locations that can store and communicate data with other components of the system 100. The server 110 can include a database 111 that stores a plurality of performance tests or tasks 112.

The tasks 112 include computer executable instructions that enable the system 100 to administer a task to a user, obtain performance information captured via one or more sensors (e.g., a camera, microphone, etc.), and then enable the server 110 to analyze the performance of the test and determine whether a condition may exist.

For the example task 112 above, the database 111 stores the executable instructions that enables the presentation of instructions via the virtual agent 121 (which could be a video of someone performing the test), the capturing of the patient performing the test (such as via a video camera on the computing device 120), the analysis of the task to determine a condition (in this case, level of impairment) and the transmission of the test to appropriate parties (the patient themselves, health care providers, etc.).

The data and instructions associated with a task 112 can include one or more metrics that are associated with the task 112 that can give an indication of the potential presence of autism spectrum disorder or symptoms thereof and the severity. The metrics can be thought of as the measurable characteristics associated with the user's performance of the task that have been observed to be related or correlated with autism spectrum disorder. The metrics thus could be considered attributes, whose values can be measured by the system when the user performs a task. The data and instructions of task 112 can also include one or more thresholds of values, beyond which (above or below, depending on the metric) the metric can be considered to be indicative of the presence of autism spectrum disorder (alone or in combination with other metrics).

Impaired lip rounding is a characteristic feature in autism due to persistent apraxia of speech. Therefore, many of the tasks will involve tasks to extract speech and facial biomarkers. However, other types of biomarkers are also used such as biomarkers associated with memory encoding, storage, retrieval and attention.

Examples of tasks 112 can include one or more of a counting task (e.g., counting up from one on a single breath), a reading task (e.g., reading sentences, reading consonant-vowel-consonant words, etc.), an oral diadochokinesis task (e.g., alternating motion rate, repetition of certain syllables, etc.), a picture description task, a spontaneous speech task, a forward-and-backward digit span task, a word recall task, a semantic fluency task (e.g., name all animals you can think of), a motor task, an emotion elicitation task, or a sequential command task.

The client computing devices 120 can access the functions of the inventive subject matter via multiple ways. For example, a downloadable application or via a web portal accessible over a browser. The client computing devices 120 include at least one processor, at least one non-transitory computer-readable storage medium, and I/O interfaces that allow a user to receive data from and interact with the computing device 120 (e.g., monitor, touch screen, speakers, mouse, keyboard, cameras, etc.). The client computing devices 120 also have communication interfaces (e.g., Wi-Fi, wired internet connection, cellular, etc.) that enable the device 120 to exchange data over network 130. Examples of suitable computing devices 120 can include desktop computers, laptop computers, tablets, smartphones, and video game consoles.

To administer the tasks 112 and enable other interactions with a patient, a client computing device 120 executes a virtual agent 121. The virtual agent 121 can be installed on the client computing device 120. In other embodiments, the virtual agent 121 is executed by the server 110 and merely presented on the client computing device 120 via a web browser or other user-facing portal.

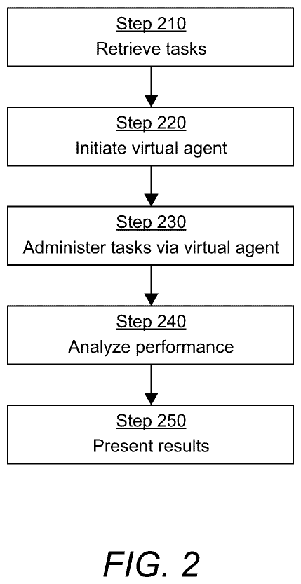

FIG. 2 is a flowchart of a process according to embodiments of the inventive subject matter.

At step 210, the server 110 retrieves one or more tasks that are to be presented to the user for performance. The retrieval of the tasks, including the selection of one or more of the tasks, can be based on a previous recommendation or instruction, such as by the user's physician or other medical professional.

At step 220, the computing device 120 initiates the virtual agent 121. The initiation of the virtual agent 121 can be in response to the server 110 identifying that one or more performance tests need to be administered to this specific user. The virtual agent 121 can, in embodiments, be initialized based on a user logging on to their account and accessing a test.

At step 230, the server 110 executes the task such that it is administered via the virtual agent 121 on computing device 120.

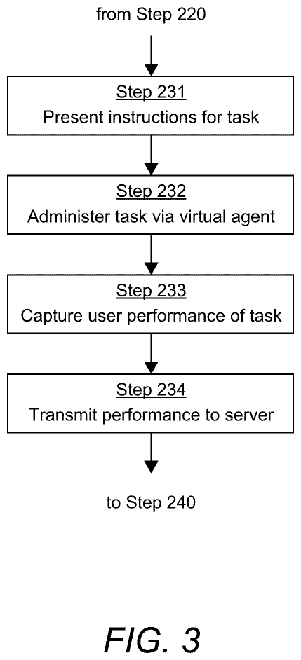

The detailed steps of the administration of the task can be seen in FIG. 3.

Via the virtual agent 121, the administration of the task can include presenting instructions for the task at step 231. This can include visual and/or audio instructions presented via the virtual agent 121 that explains the task to the user. The visual components of the instructions can include text, still images and/or video images.

For example, the instructions for a task involving reading sentences or certain words can include a video that shows the virtual agent 121 presenting the text and then a person reading the text back. The instructions can also include a prompt to begin the task.

The instructions can also include directions to the user regarding camera placement, framing of the camera and proper positioning relative to the camera.

At step 232, the server 110 administers the task via the virtual agent 121. The administration of the task via the virtual agent 121 can vary depending on the task itself.

To begin the administration of the task, the virtual agent 121 can ask the user to click on a “ready” button or speak a word indicating they are ready. In embodiments, the virtual agent 121 can have a countdown or other indication that the task will begin shortly after the instructions of step 231 so that the user does not have to interact with the system at all to transition into the task.

At step 233, the computing device 120 captures the user's performance of the task 112 via one or more sensors 122 integral or connected to the computing device 120. In preferred embodiments, the sensors 122 include a camera and a microphone (which can be integral to or separate from the camera). Other peripherals used by the user to perform the task can include a touchscreen, a keyboard or mouse, etc.

The sensor data captured by the sensor 122 during the performance of the task can be transmitted to the server 110 by the computing device 120 at step 234. In preferred embodiments, the sensor data is audiovisual data. The sensor data can be streamed to the server 110 or otherwise transmitted to the server 110 (such as by first saving on the computing device 120 and then transmitting it to the server 110). The process then moves on to step 240.

At step 240, the server 110 analyzes the performance of the task based on the sensor data captured by sensor 122.

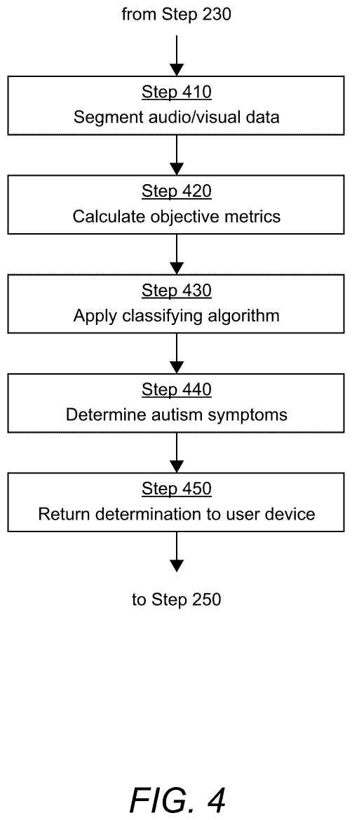

The analysis of step 240 is shown in detail in the flowchart of FIG. 4.

At step 410, the server 110 segments the audiovisual data. In embodiments of the inventive subject matter, segmenting the audiovisual data can include the server 110 segmenting the audiovisual data into individual utterances (e.g., words, sentences, phrases or a section of speech) and/or video captures of the user. The segmenting can be performed at various levels of granularity. For example, the segmentation can be a segmentation of the audio/video stream into “dialog turns” (i.e., segmenting into the prompts of the virtual agent and then the responses of the participant). The response segments can then be segmented into individual utterances (e.g., words, sentences, phrases or sections of speech).

At step 420, the server 110 uses the segmented audiovisual data to calculate objective metrics for the user based on the user's performance of the task. The objective metrics calculated by the server 110 can include metrics in the following domains: a speech acoustic domain, a facial domain, a linguistic domain, a cognitive domain, a motor domain, and an emotional domain. The methods employed by the server 110 to derive the objective metrics can depend on the particular domain for which the objective metrics are being calculated. The speech acoustic domain can include metrics derived from an analysis of the audio signal itself. The facial domain can include metrics derived from analysis of facial landmarks (i.e., points of interest on the face) in the video signal. The linguistic domain can include metrics derived from analysis of the spoken words themselves, based on speech-to-text technologies and then applying natural language processing (“NLP”) methods to the textual transcriptions. The cognitive domain can include metrics representative of results of analysis of cognitive and memory abilities. The metrics for the cognitive domain can often stem from or be derived from metrics associated with the other modalities. The Motor domain refers to measurements of properties of movements of different body parts, e.g., limbs, fingers, hands (e.g., in fingertapping tasks), orofacial movement (like movement of lips and raising of eyebrows), etc., and the metrics are representative of these movements. The emotional domain refers to affective features that capture various emotions—examples are smiles or frowns on the face, or raised or lowered pitch for voice. The objective metrics for the emotional domain are those that are reflective of the affective features as captured from the user. The underlying tasks that result in the metrics used for the cognitive domain metrics are specific to cognitive testing.

To extract metrics in the speech acoustic domain, the server 110 must first be able to recognize the spoken speech. To do so, the server 110 applies known acoustic and vocal recognition techniques. To extract metrics in the visual/facial domain, the server 110 must first be able to recognize or understand an image. To do so, the server 110 can apply known image recognition techniques. For metrics in the facial domain, the server 110 derives the metrics based on facial landmarks that are extracted using known face detectors and facial landmark detectors.

The server 110 derives linguistic metrics using known linguistic and/or transcription software services, which includes software that computes lexico-semantic features for the spontaneous speech parts of the conversation.

For the cognitive score associated with cognitive tasks (such as word and digit recall), the server 110 first applies voice recognition and/or linguistic/transcription software services to recognize the spoken words. The server 110 then applies a scoring based on this recognition. For example, for word recall tasks, a score can be a percentage of correct words. For digit span tasks, the server 110 can score the task based on whether the digits were repeated in the same order (for example, a score of “2”), whether all digits were present, but not in the correct order (a score of “1”) or that not all digits were recited (a score of “0”).

For visual metrics, the server 110 can measure pixel distance to account for a distance of movement of relevant features (jaw, lips, etc., depending on the task and/or metric).

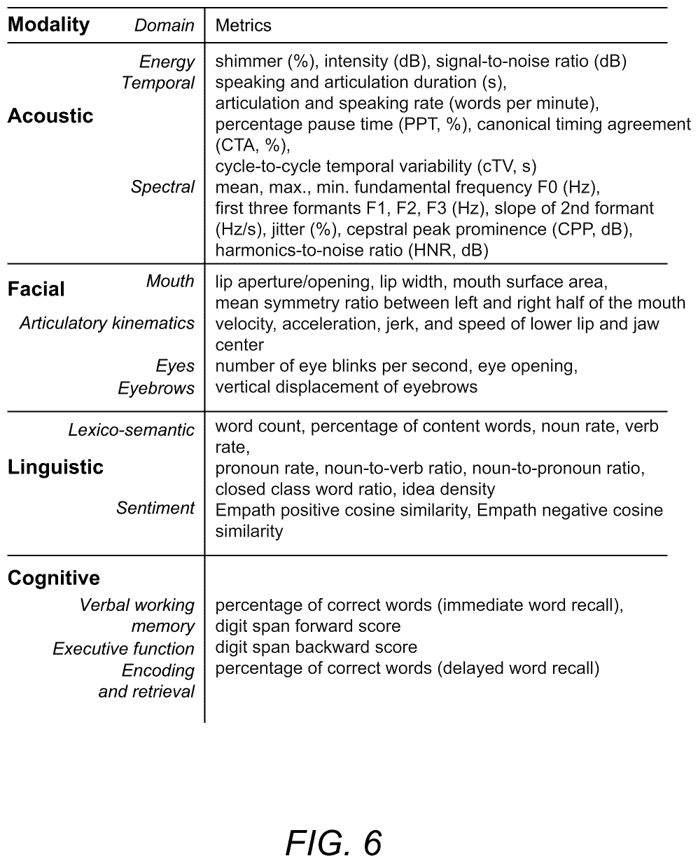

FIG. 6 shows a table with some example metrics that can be derived by the server 110. In the table of FIG. 6, the metrics are divided into categories/modalities of acoustic, facial, linguistic, and cognitive. Within these are examples of domains having in each modality and the corresponding metrics that are observable/derivable within the domains for each modality.

In embodiments of the inventive subject matter, the server 110 can merge demographic information about the user with the objective metrics at step 420. The demographic information can include information such as age, country of birth, employment status, ethnicity, first language, gender, relationship/marital status, sex, student status, etc.

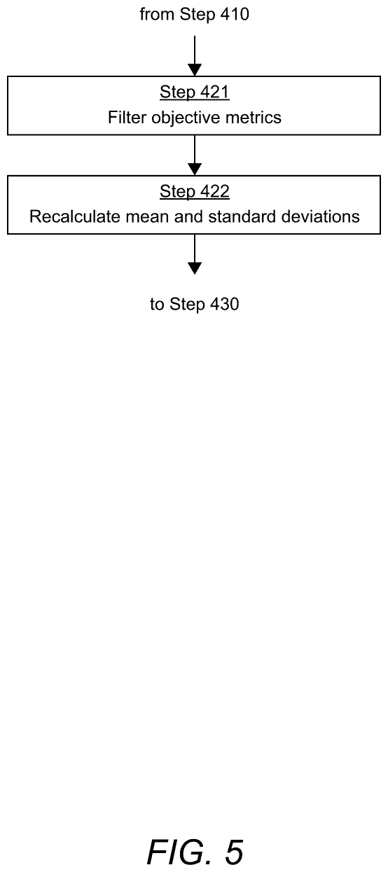

In embodiments of the inventive subject matter, the server 110 can refine the objective metrics after they are calculated. The process of refining the objective metrics is seen on FIG. 5, and is as follows:

At step 421, the server 110 first removes any objective metrics values beyond a predefined number of standard deviations. For example, in this process, the predefined number of standard deviations is five standard deviations though other numbers of standard deviations are contemplated.

At step 422, the server 100 recalculates a mean and a standard deviation for the remaining objective metrics from step 421, and then removes any remaining objective metrics having values beyond a second predefined number of standard deviations. For this example, the second predefined number of standard deviations is three standard deviations. Other numbers of standard deviations are also contemplated.

At step 430, the server 110 applies a classifying algorithm to the objective metrics.

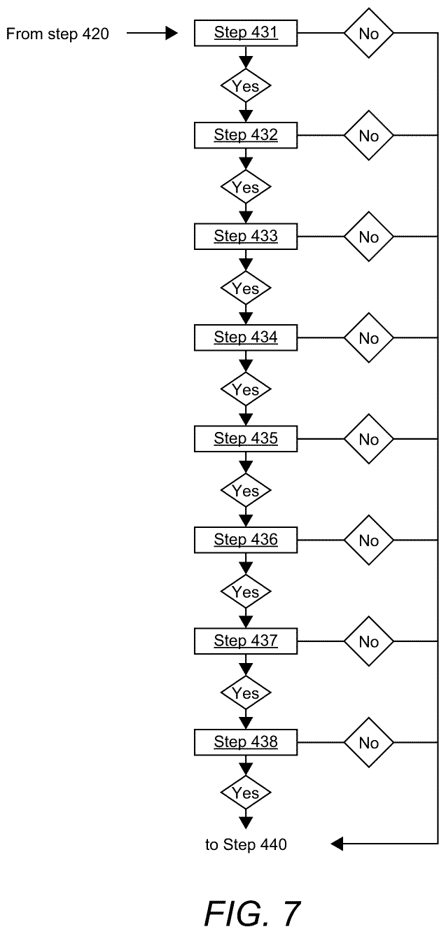

In embodiments of the inventive subject matter, the classifying algorithm is executed is illustrated on FIG. 7 as follows:

At step 431 the server 110 determines whether the third formant frequency value (F3 in Hz) during productions of the vowel /u/ is greater than a threshold value (in Hz). If so, the server 110 continues to step 432.

At step 432, the server 110 determines whether the third formant frequency value (F3 in Hz) during productions of the vowel /ε/ is greater than a corresponding threshold value in Hz. If so, the server 110 continues to step 433.

At step 433, the server 110 determines whether the cycle-to-cycle temporal variability during oral diadochokinesis is greater than a threshold value in seconds. If so, the server 110 continues to step 434.

At step 434, the server 110 determines whether the percentage pause time while counting up from one on a single breath is greater than a threshold percentage value. If so, the server 110 continues to step 435.

At step 435, the server 110 determines whether the maximum jaw speed while producing the vowel /α/ is slower than a threshold value. If so, the server 110 continues to step 436.

At step 436, the server 110 determines whether the delayed recall of words score is less than a threshold value. If so, the server 110 continues to step 437.

At step 437, the server 110 determines whether the ratio of closed class or function words (prepositions, conjunctions, etc.) to open class words during picture description is less than a threshold value. If so, the server 110 continues to step 438.

At step 438, the server 110 determines whether the positive cosine similarity during spontaneous speech production is greater than a threshold value. If so, the server 110 determines that the user is exhibiting autism symptoms and is likely to have autism spectrum disorder at step 440.

If any of the thresholds at any of the steps 431-438 are not met, the server 110 continues directly to step 440 and determines that the user is unlikely to exhibit autism symptoms or have autism spectrum disorder.

In embodiments of the inventive subject matter, the server 110 can transmit the determination and other information to the computing device(s) of one or more healthcare providers at step 250.

It is contemplated that other algorithms can be applied to the objective metrics to determine whether a user exhibits autism symptoms or likely has autism spectrum disorder.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method for detecting a possible presence of autism, comprising:
presenting, by a computing device and via a virtual agent, at least one task for a user to perform;
capturing, by the computing device, audiovisual data of the performance of the at least one task by the user;
streaming, by the computing device, the audiovisual data to at least one remote computing device;
segmenting, by the remote computing device, the audiovisual data;
calculating, by the remote computing device, objective metrics for the user;
applying, by the remote computing device, a classifying algorithm to the objective metrics; and
determining, by the remote computing device, whether the user has autism spectrum disorder based on an output of the classifying algorithm.

2. The method of claim 1, wherein the objective metrics are derived according to one or more of a speech acoustic domain, a facial domain, a linguistic domain and a cognitive domain.

3. The method of claim 1, wherein the at least one task comprises at least one of a counting task, a consonant-vowel-consonant reading task, an oral diadochokinesis task, a reading task, a picture description task, a reading sentences task, a spontaneous speech task, a forward-and-backward digit span task, a word recall task, a semantic fluency task and a sequential command task.

4. The method of claim 1 further comprising, after the step of calculating: merging, by the remote computing device, demographics information with the calculated objective metrics.

5. The method of claim 1, further comprising refining the objective metrics following the step of calculating, wherein the step of refining the objective metrics comprises:
removing, by the remote computing device, any objective metrics beyond five standard deviations; and
for remaining objective metrics, recalculating, by the remote computing device, a mean and a standard deviation and removing any remaining objective metrics with values beyond three standard deviations.

6. The method of claim 1, applying the classifying algorithm further comprises:
a. determining, by the remote computing device, if the third formant frequency value (F3 in Hz) during productions of the vowel /u/ greater than a predetermined amount of Hz, go to step b, else go to step j;
b. determining, by the remote computing device, if the third formant frequency value (F3 in Hz) during productions of the vowel /ε/ greater than a predetermined amount of Hz, go to step c, else go to step j;
c. determining, by the remote computing device, if the cycle-to-cycle temporal variability during oral diadochokinesis is greater than a predetermined amount (seconds), else go to step j;
d. determining, by the remote computing device, if the percentage pause time while counting up from one on a single breath is greater than a predetermined percentage amount (%), go to step e, else go to step j;
e. determining, by the remote computing device, if the maximum jaw speed while producing the vowel /α/ is slower than a predetermined amount, go to step f, else go to step j;
f. determining, by the remote computing device, If the delayed recall of words score is less than a predetermined amount, go to step g, else go to step j;
g. determining, by the remote computing device, if the ratio of closed class or function words (prepositions, conjunctions, etc.) to open class words during picture description is less than a predetermined amount, go to step h, else go to step j;
h. determining, by the remote computing device, if the positive cosine similarity during spontaneous speech production is greater than a predetermined amount, go to step i, else go to step j;
i. determining, by the remote computing device, that the user may have autism spectrum disorder; and
j. determining, by the remote computing device, that the user is unlikely to have autism spectrum disorder.

7. The method of claim 6, further comprising:
providing, by the remote computing device, the output of step (i) or (j) to the computing device; and
presenting, by the computing device, the output to the user.

8. The method of claim 6, further comprising providing, by the remote computing device, the output of step (i) to a healthcare provider.
